(19) United States
(12) Patent Application Publication
(10) Pub. No.: US 2026/0091218 A1
(43) Pub. Date: Apr. 2, 2026

(54) CAGE FOR RIGHT-SIDED CARDIAC PUMP

(71) Applicant: ABIOMED, INC., Danvers, MA (US)

(72) Inventors: Charles PERKINS, Danvers, MA (US); Cam LOUGHLIN, Danvers, MA (US); James MELTON, Danvers, MA (US); Jairo Leandro MOUX, Danvers, MA (US); Frank KIRCHHOFF, Danvers, MA (US); Thorsten SIESS, Danvers, MA (US); Nandish DESAI, Danvers, MA (US)

(73) Assignee: ABIOMED, INC., Danvers, MA (US)

(21) Appl. No.: 19/344,060

(22) Filed: Sep. 29, 2025

Related U.S. Application Data
(60) Provisional application No. 63/701,151, filed on Sep. 30, 2024.

(51) Int. Cl. A61M 60/81; A61M 60/13; A61M 60/165; A61M 60/216; A61M 60/414; A61M 60/90

(52) U.S. Cl. CPC A61M 60/81; A61M 60/13; A61M 60/165; A61M 60/216; A61M 60/414; A61M 60/90; A61M 2205/103; A61M 2205/3334; A61M 2205/50; A61M 2205/7545; A61M 2210/125

(57) ABSTRACT

Methods of and systems for performing a medical procedure are herein disclosed. The presently disclosed system generally includes a catheter body, a pump assembly, a cannula, one or more inlet ports, one or more outlet ports, and a cage. The cage is connected to the cannula and the pump assembly, extending along the longitudinal axis and in a circumferential direction about the longitudinal axis surrounding the one or more cannular inlet ports. The cage includes a first section, a second section, and a plurality of cage inlet ports. The pump assembly is operable to cause fluid to flow (i) through a first plurality of cage inlet ports at a first flow rate and through a second plurality of cage inlet ports at a second flow rate different from the first flow rate, (ii) through the one or more inlet ports, and (iii) through the one or more outlet ports.

### CROSS-REFERENCE TO RELATED APPLICATIONS

This application claims the benefit of priority under 35 U.S.C. § 119 to prior filed U.S. Provisional Patent Application No. 63/701,151, filed Sep. 30, 2024 (Attorney Docket No.: 266489.000005 (ABD0349USPSP1/D0349.US01)), the entire contents of which is hereby incorporated by reference as if set forth in full herein.

### FIELD

The present disclosure relates generally to medical devices, and in particular to catheters. More specifically, this disclosure relates to a blood pump, in particular an intravascular blood pump for percutaneous insertion into a patient's blood vessel, to support a blood flow in a patient's blood vessel. This disclosure particularly relates to a right ventricular blood pump to support a blood flow from the vena cava through the right ventricle into the pulmonary artery.

### BACKGROUND

Intravascular blood pumps are inserted into a patient's vessel such as the aorta or vena cava and through a cardiac valve by means of a catheter and can also be referred to as catheter pumps. A blood pump typically comprises a pump section with a blood flow inlet and a blood flow outlet. To cause blood flow from the blood flow inlet to the blood flow outlet, typically an impeller or rotor is rotatably supported within the pump casing about an axis of rotation for conveying blood. The blood pump may be driven by a motor included in the blood pump adjacent to the pump section or may alternatively be driven by a motor outside the patient's body, in which case the motor is connected to the impeller by a flexible drive shaft extending through the catheter.

A right ventricular blood pump is inserted through the inferior or superior vena cava through the right ventricle of a patient's heart into the pulmonary artery by means of a catheter. Typically, the blood flow inlet of the blood pump is placed inside the right atrium, vena cava or right ventricle, while the pump section extends through the tricuspid valve, the right ventricle and the pulmonary valve into the pulmonary artery.

Any blood clots or thrombi that occur may be conveyed from the vena cava to the pulmonary artery, which, however does not cause severe harm to the patient because the thrombi only end up in the pulmonary circulation. More importantly, blood clots may clog the blood pump and thus may cause failure of the blood pump, which has to be avoided. Filters are known that can be placed in the vena cava, such as balls of random Nitinol wires. However, handling of such filters adjunctively to the blood pump therapy may be cumbersome, e.g., their insertion and particularly removal would require an additional access point. Any access point carries a risk of bleeding and infection. A correct alignment between blood pump and filter is necessary for performance and needs to be confirmed by means of visualization by echo or x-ray.

### SUMMARY

The disclosed technology includes a catheter for insertion into a patient's vasculature, the catheter extending along a longitudinal axis. The catheter comprises a catheter body, a pump assembly, a cannula, one or more inlet ports, one or more outlet ports, and a cage. The catheter body comprises a distal end and a proximal end. The pump assembly is disposed at the distal end of the catheter body and comprises a distal end portion. The cannula is connected to the distal end portion of the pump assembly and comprising a proximal cannula portion and a distal cannula portion. The one or more inlet ports are disposed proximal the proximal cannula portion. The one or more outlet ports are disposed proximal the distal cannula portion. The cage is connected to the cannula and the pump assembly, extends along the longitudinal axis and in a circumferential direction about the longitudinal axis surrounding the one or more cannular inlet ports. The cage comprises a first section and a second section connected to the first section along the longitudinal axis, the first section being spaced from the one or more cannula input ports along the longitudinal axis and the second section being disposed over the one or more inlet ports in a radial direction, and a plurality of cage inlet ports comprising a first plurality of cage inlet ports disposed in the first section of the cage and a second plurality of cage inlet ports disposed in the second section of the cage. The pump assembly is operable to cause fluid to flow (i) through the first plurality of cage inlet ports at a first flow rate and through the second plurality of cage inlet ports at a second flow rate different from the first flow rate, (ii) through the one or more inlet ports, and (iii) through the one or more outlet ports.

The disclosed technology includes catheter for insertion into a patient's vasculature, the catheter extending along a longitudinal axis. The catheter comprises a catheter body, a pump assembly, a cannula, one or more inlet ports, one or more outlet ports, and a cage. The catheter body comprises a distal end and a proximal end. The pump assembly is disposed at the distal end of the catheter body and comprises a distal end portion. The cannula is connected to the distal end portion of the pump assembly and comprising a proximal cannula portion and a distal cannula portion. The one or more inlet ports are disposed proximal the proximal cannula portion. The one or more outlet ports are disposed proximal the distal cannula portion. The cage is connected to the cannula and the pump assembly, extends along the longitudinal axis and in a circumferential direction about the longitudinal axis surrounding the one or more cannular inlet ports, and comprises a first section and a second section connected to the first section along the longitudinal axis, the first section being spaced from the one or more cannula input ports along the longitudinal axis and the second section being disposed over the one or more inlet ports in a radial direction, and a plurality of cage inlet ports. The cage inlet ports comprise a first plurality of cage inlet ports disposed in the first section of the cage, with each of the first plurality of cage inlet ports comprising a first area, and a second plurality of cage inlet ports disposed in the second section of the cage, with each of the second plurality of cage inlet ports comprising a second area. The pump assembly is operable to cause fluid to flow (i) through the first plurality of cage inlet ports and through the second plurality of cage inlet ports, (ii) through the one or more inlet ports, and (iii) through the one or more outlet ports.

The disclosed technology includes a medical system comprising a catheter and a controller. The catheter extends along a longitudinal axis and comprises a catheter body, a pump assembly, a cannula, one or more inlet ports, one or more outlet ports, and a cage. The catheter body comprises a distal end and a proximal end. The pump assembly is disposed at the distal end of the catheter body and comprises a distal end portion. The cannula is connected to the distal end portion of the pump assembly and comprising a proximal cannula portion and a distal cannula portion. The one or more inlet ports are disposed proximal the proximal cannula portion. The one or more outlet ports are disposed proximal the distal cannula portion. The cage is connected to the cannula and the pump assembly, and comprises a plurality of cage inlet ports, with the cage extending along the longitudinal axis and in a circumferential direction about the longitudinal axis. The controller is connected to the proximal end of the catheter body and is configured to operate the pump assembly to cause fluid to flow through the cage inlet ports, through the one or more inlet ports, and through the one or more outlet ports.

The disclosed technology includes a method of performing a medical procedure. The method comprises navigating a catheter to a target location in a heart of a patient, the catheter extending along a longitudinal axis and comprising (i) a catheter body, (ii) a pump assembly connected to the catheter body, (iii) a cannula connected to the pump assembly and comprising one or more inlet ports, and (iv) a cage connected to the cannula and the pump assembly, the cage comprising a plurality of sections, includes a first section and a second section connected to the first section, and a plurality of cage inlet ports. The method comprises operating the pump assembly to: pull blood through each cage inlet port and the one or more inlet ports such that (i) any blood pulled through the one or more inlet ports is first pulled through one of the cage inlet ports and (ii) the second section of the cage either reduces a flow rate of the blood relative to the first section or prevents blood from flowing through the second section. The method comprises capturing a biomaterial at one of the cage inlet ports.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements. The figures depict one or more implementations of the inventive devices, by way of example only, not by way of limitation.

FIG. 1 is a schematic pictorial illustration of a catheter inserted into a patient's heart, in accordance with the disclosed technology;

FIG. 2 is a schematic pictorial illustration of a medical system including the catheter of FIG. 1 including a cage, with the cage being shown in cross-section, in accordance with the disclosed technology;

FIG. 3 is a schematic pictorial illustration of a detail view of Detail A in FIG. 2, in accordance with the disclosed technology;

FIG. 4A is a schematic pictorial illustration of an elevation view of the cage, in accordance with the disclosed technology;

FIG. 4B is a schematic pictorial illustration of a detail view of Detail B in FIG. 4A, in accordance with the disclosed technology;

FIG. 4C is a schematic pictorial illustration of a detail view, similar to FIG. 4B, depicting an alternative configuration of the cage;

FIG. 5 is a schematic pictorial illustration of a section view of the cage and inlet ports, taken perpendicular to a longitudinal axis of the catheter, in accordance with the disclosed technology;

FIG. 6A is a schematic pictorial illustration of an elevation view of an alternative cage configuration, in accordance with the disclosed technology;

FIG. 6B is a schematic pictorial illustration of an elevation view of an alternative cage configuration, in accordance with the disclosed technology;

FIG. 6C is a schematic pictorial illustration of an elevation view of an alternative cage configuration, in accordance with the disclosed technology;

FIG. 6D is a schematic pictorial illustration of an elevation view of an alternative cage configuration, in accordance with the disclosed technology;

FIG. 7A is a schematic pictorial illustration, similar to FIG. 3, showing the catheter in use, in accordance with the disclosed technology;

FIG. 7B is a schematic pictorial illustration, similar to FIG. 3, showing the catheter in use, in accordance with the disclosed technology;

FIG. 8 is a flow chart of a method of using a catheter, according to aspects of the present disclosure;

FIG. 9A is a schematic pictorial illustration of a block diagram depicting an exemplary sensor and cage arrangement, according to aspects of the present disclosure;

FIG. 9B is a schematic pictorial illustration of a block diagram depicting another exemplary sensor and cage arrangement, according to aspects of the present disclosure;

FIG. 9C is a schematic pictorial illustration of a block diagram depicting another exemplary sensor and cage arrangement, according to aspects of the present disclosure; and

FIG. 9D is a schematic pictorial illustration of a block diagram depicting another exemplary sensor and cage arrangement, according to aspects of the present disclosure.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%.

In addition, as used herein, the terms “patient,” “host,” “user,” and “subject” refer to any human or animal subject and are not intended to limit the systems or methods to human use, although use of the subject invention in a human patient represents a preferred embodiment. As well, the term “proximal” indicates a location closer to the operator whereas “distal” indicates a location further away to the operator or physician.

As discussed herein, the terms “tubular” and “tube” are to be construed broadly and are not limited to a structure that is a right cylinder or strictly circumferential in cross-section or of a uniform cross-section throughout its length. For example, the tubular structures are generally illustrated as a substantially right cylindrical structure. However, the tubular structures may have a tapered or curved outer surface without departing from the scope of the present disclosure.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

The disclosed technology relates to a catheter (e.g., an intravascular pump) that is configured to trap biomaterial prior to its entry into a pump assembly. More specifically, a cage surrounds the pump assembly upstream and downstream relative to the native directional flow of blood in the heart. The design optimizes suction of the pump assembly across all inlet surfaces of the cage, but maximizes flow velocity and magnitude of negative pressure at windows in the cage to enable a predictable capture of biomaterial at the predetermined location and prevention of its entry into the catheter, which can reduce or inhibit its operation.

To help the reader better understand the disclosed technology, the catheter will be described, in general, first and then various devices/methods for capturing biomaterial will be described in relation to the intravascular pump. It will be appreciated, however, that the disclosed technology can be applicable to other intravascular pumps having differing designs from that described herein. Furthermore, the disclosed technology can be applicable to other catheters in which entry of biomaterial into a pump is common. Accordingly, the disclosed technology should not be limited to the specific catheter shown in the drawings and described herein.

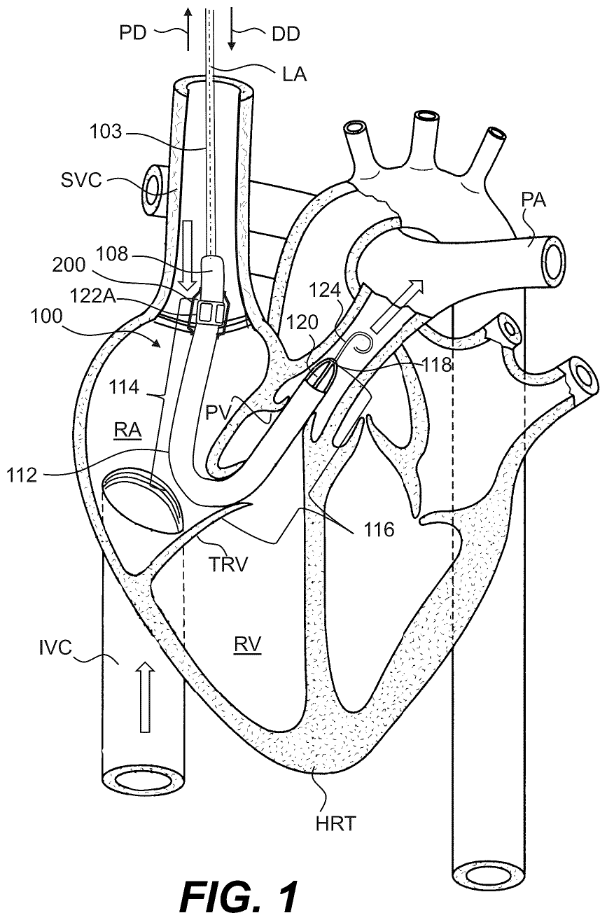

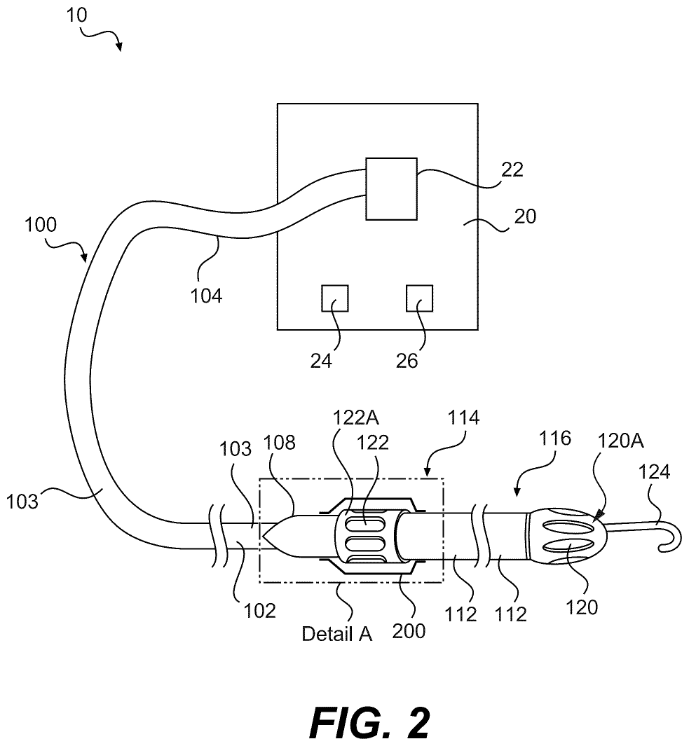

FIG. 1 illustrates a catheter 100 (which can be embodied as, e.g., an intravascular blood pump) inserted into a patient's heart HRT, while FIG. 2 illustrates a medical system 10 that includes the catheter 100 and a controller 20. With reference to FIGS. 1 and 2, the catheter 100 includes a catheter body 103, a pump assembly 108, and a flexible cannula 112. The catheter body 103 extends along a longitudinal axis LA from a proximal end 104 to a distal end 102 (labelled in FIG. 2). The pump assembly 108 is coupled to the distal end 102 of the catheter body 103 and the controller 20 is coupled to the proximal end 104 of the catheter body 103. The cannula 112 is coupled to a distal end portion 110 of the pump assembly 108. The cannula 112 has a proximal cannula portion 114 and a distal cannula portion 116 and defines a lumen 112A. The proximal cannula portion 114 and distal cannula portion 116 may be approximately parallel to the longitudinal axis LA of the catheter body 103 and may be approximately straight prior to delivery into the patient's heart HRT.

The catheter 100 further includes one or more inlet ports 122 (which can be a part of or separate from the cannula 112) disposed on or near the proximal cannula portion 114 and one or more outlet ports 120 disposed on or near the distal cannula portion 116 at a distal end of the catheter 100. In the present example, the blood flow inlet port(s) 122 and the blood flow outlet port(s) 120 are formed as circumferential openings in respective cages 122A, 120A. It will be appreciated that other shapes, sizes or positions may be suitable for the inlet ports 122 and outlet ports 120, possibly depending on the application. In the present example, the cages 120A, 122A are separate from the cannula 112; however, it will be appreciated that, in other examples, the ports 120, 122 can be defined integrally with the cannula 112.

The shape, size, and material of the cannula 112 is optimized for insertion of the catheter 100 into the right side of the heart of the patient such that the cannula 112 traverses the superior vena cava SVC, right atrium RAs tricuspid valve TRV, right ventricle RV, and pulmonary valve PV of the heart HRT. This allows the inlet ports 122 to be positioned in the superior vena cava SVC, the inferior vena cava, or the right atrium RA and the outlet ports 120 to be positioned in the pulmonary artery PA.

In some implementations, the length of the cannula 112 is 22 cm. In certain implementations, the length of the cannula may be in the range of 17-25 cm or any other suitable length. The cannula 112 is constructed to allow fluid to flow into the inlet ports 122, through the cannula 112, and out the outlet ports 120. The fluid may be propelled through the cannula 112 by an impeller 109 of the pump assembly 108 (discussed in greater detail below) located in the pump assembly 108. In some examples, the cannula 112 may be configured to be relatively stiff to increase the stability of the cannula 112 once in place in the right heart.

The cannula 112 is also sized for passage through a femoral artery and other vasculature of a patient. In some implementations, the cannula 112 has a cannula diameter of about 22 Fr. In certain implementations, the cannula 112 may have a cannula diameter of 7 Fr, 8 Fr, 9 Fr, 10 Fr, 11 Fr, 12 Fr, 18 Fr, 20 Fr, 21 Fr, 22 Fr, 23 Fr, 24 Fr, or any other suitable diameter. The cannula diameter may be approximately constant along the length of the cannula 112.

In certain implementations, the cage 120A defining the outlet ports 120 of the catheter 100 may narrow toward a distal end of the outlet ports 120, which can further facilitate passage through the heart valves. In certain implementations, a flexible extension 124 can be connected to the outlet ports 120 to prevent traumatic contact of the distal end of the catheter 120 with interior walls of the heart following insertion.

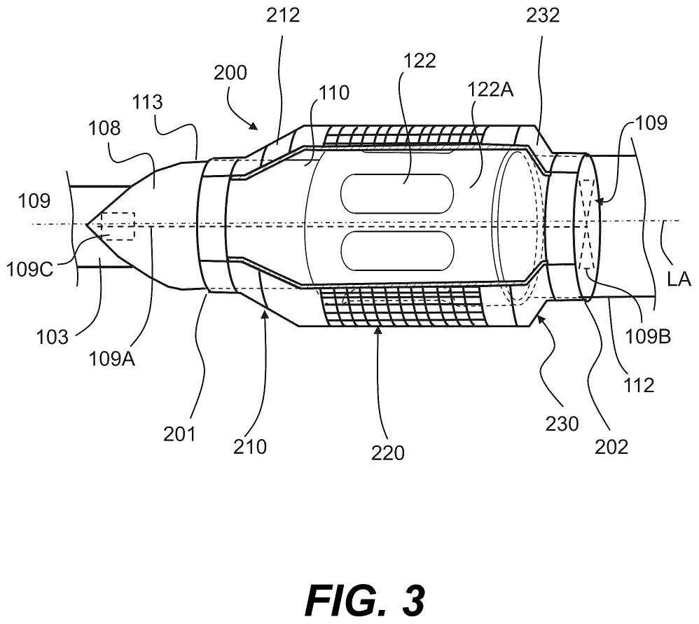

As seen in phantom lines in FIG. 3, the pump assembly 108 can contain a rotor 109A which may be driven by an implantable or external drive unit. In some examples, the pump assembly includes an impeller 109 having (i) the rotor 109A, with the rotor 109A being connected to the distal end 102 of the catheter body 103, (ii) one of more impeller blades 109B connected to and rotatable by tie rotor 109A, and (iii) a motor 109C connected to the rotor 109A that causes rotation thereof. The pump assembly 108 includes a housing 113 which may be comprised of a different material than the cannula 112 or catheter 103. The rotor 109A of the pump assembly 108 may be located in the housing 113 and attached to a drive shaft (not shown). Although an example with an implantable motor 109C is shown in FIG. 3, in some implementations the unit driving the drive shaft is located external to the patient's body and the drive shaft extends through the catheter body 103. In some implementations, a motor 109C driving the drive shaft is enclosed within the pump assembly 108 (as shown). However, any suitable pump and/or drive known in the art may be used.

The pump assembly 108 is configured to provide a fluid flow into the cannula 112 at the inlet ports 122, through the cannula 112, and out the outlet ports 120. The pump assembly 108 may be configured to provide a flow rate of 4 liters per minute (lpm) or more within the right heart of a patient. In some implementations, the pump assembly 108 may provide a flow rate of 3 lpm, 3.5 lpm, 4 lpm, 4.5 lpm 5 lpm, 6 lpm or any other suitable flow rate. In some implementations, the flow rate is chosen based on the needs of the patient.

The controller 20 is functional to control operation of the pump assembly 108. The controller 20 includes a fluid reservoir 22 that is configured to contain a purge fluid for purging the motor 109C, which prevents buildup on the rotor 109A and/or impeller 109B and also cools the motor 109C. In some examples, the purge fluid includes heparin, sodium bicarbonate, or any other appropriate fluid and/or combinations thereof in various proportions. The controller 20 is configured to deliver the purge fluid through the pump assembly and/or the motor 109C. The controller 20 can include a fluid supply control 24 for controlling supply of the purge fluid to the pump assembly and a flow rate control 26 for controlling operation of the pump assembly 108. In operation, the controller 20 controls the pump assembly 108 to pull a fluid (e.g., blood) through the inlet ports 122, through the lumen 112A of the cannula 112 and expel the fluid through the outlet ports 120.

Further to the above and as seen in FIG. 1, in use, the catheter 100 is inserted into the right ventricle. RV of the patient's heart HRT in a distal direction DD via the superior vena cava SVC and right atrium RA (as shown). In a different approach, the catheter may be inserted: through the inferior vena cava IVC. During its operation, cannula 112 placed through the tricuspid valve TRV and the pulmonary valve PV. The pump assembly 108 via the impeller 109 (FIG. 3), causes the blood to flow into the inlet ports 122 towards and out of the outlet ports 120 (see the arrows in FIG. 1), To remove and/or adjust a position of the catheter 100, it can be moved in a proximal direction PD.

In some instances, while the pump assembly 108 is being operated, biomaterial (e.g., thrombus) may undesirably enter through the inlet ports 122, which may become trapped in and/or occlude the motor 109C and/or on the impeller blades 109B. This may cause a significant decrease in the flow rate of the pump assembly 108. It is desirable, therefore, to provide for the capture of the biomaterial prior to entry through the inlet ports 122. In examples detailed below in the present disclosure, this is achieved via a cage 200 that surrounds the inlet ports 122, with its design resulting in at least two flow rates (and/or ranges of flow rates) therethrough. In some examples, the two flow rates are different/distinct from one another. Moreover, in some examples, detection techniques (e.g., the use of optical signals) can be employed to determine when the biomaterial is captured at the predefined location.

FIG. 3 is a schematic pictorial illustration of an inlet portion of the catheter 100 that includes a cage 200. It is noted that a portion (a central portion in FIG. 3) of the cage 200 is cutaway to provide greater visibility of the underlying inlet ports 122. Moreover, it is also noted that portions of the catheter 100 hidden by the cage 200 and/or the pump assembly housing 113 are shown in phantom lines. More particularly, FIG. 3 is a detail view of Detail A in FIG. 2, with the exception of the cage 200 being shown in full (with the exception of the cutaway portion) rather than in cross-section (as in FIG. 2). With particular reference now being made to FIGS. 2 and 3, the cage 200 extends along the longitudinal axis LA and is connected to at least one of the cannula 112 and the pump assembly 108. In some examples, the cage 200 is connected (such as, but not limited to, via flow of material, adhesives, integrated welding pads, and/or any other appropriate mechanical or adhesive connection) onto the cannula 112 and/or the pump assembly 108. In some examples, the cage 200 is directly connected to the proximal portion 114 of the cannula 112 and the pump assembly 108 at opposing proximal and distal ends 201, 202 thereof. In some examples, the cage 200 includes a biocompatible metal (e.g., nickel titanium or stainless steel) or a biocompatible plastic material (e.g., flexible biocompatible plastics, such as, but not limited to, poly(methyl methacrylate) (PMMA) or thermoplastic polyurethane (TPU)). As seen best in FIG. 3, the cage 200 is dimensioned and positioned to be disposed substantially and/or entirely over the one or more inlet ports 122 along the longitudinal axis LA. Put another way, the cage 200 extends in a circumferential direction CD about the longitudinal axis to substantially surround the inlet ports 122.

The cage 200 includes at least (i) a first section 210 that comprises the proximal end 201 and (ii) a second section 220 connected to the first section 210 along the longitudinal axis. As seen in FIG. 3, the first section 210 is spaced/distanced from cannula input ports 122 along the longitudinal axis LA and the second section 220 is disposed over the one or more inlet ports 122 in a radial direction RD that is perpendicular to the longitudinal axis LA. In the presently described example, the cage 200 includes a third section 230 connected to the second section 220 along the longitudinal axis LA opposite the first section 210. As seen in FIG. 3, the third section 230 comprises the distal end 202.

The cage 200 includes a first plurality of cage inlet ports 212 disposed circumferentially around the cage 200 in the first section 210 and a second plurality of cage inlet ports 222 disposed circumferentially around the cage 200 in the second section 220. In some examples, such as that of FIG. 3, the cage can also include a third plurality of cage inlet ports 232 disposed circumferentially around the cage 200 in the third section 230.

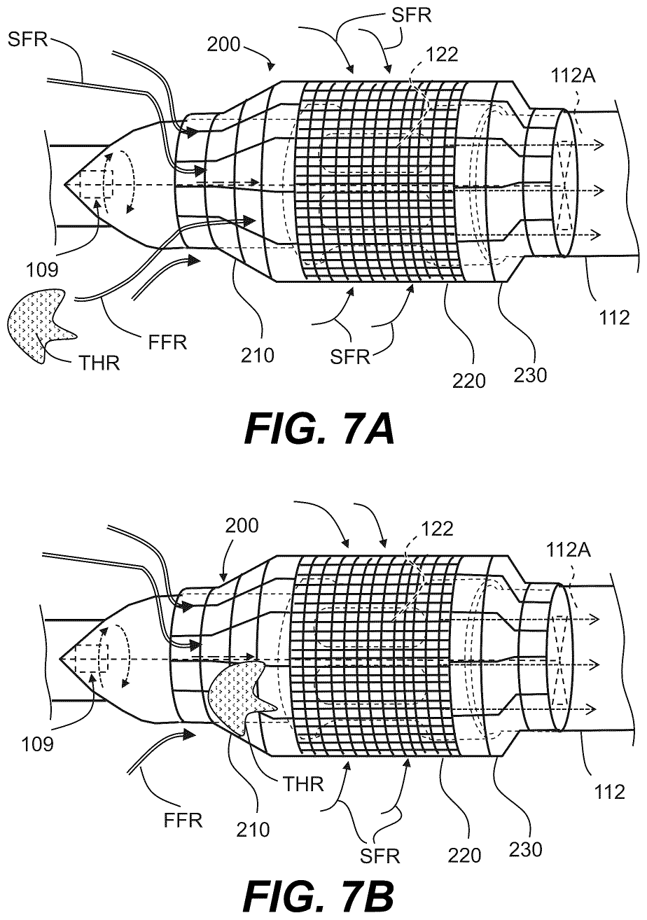

In some examples, and as discussed in greater detail below, the cage 200 is designed such that fluid flows through the first plurality of cage inlet ports 212 at a first flow rate FFR (FIG. 7A) and through the second plurality of cage inlet ports 222 at a second flow rate SFR (FIG. 7B), (ii) through the one or more inlet ports 122, and (iii) through the one or more outlet ports 120. when the pump assembly 108 is operated. In some examples, the second flow rate SFR is different from the first flow rate FFR. In some examples, the second flow rate SFR is lower from the first flow rate FFR.

It is noted that, of course, that the flow rates discussed herein may depend on a variety of external factors and may vary depending on distance from inlet ports 122, etc. For the purposes of the following description, the flow rates explicitly described are generalized to describe, e.g., a “first flow rate” that encompasses cage inlet ports with a first design, and a “second flow rate” that encompass cage inlet ports with a different design and different location along LA. In other words, and by way of example, the instantaneous flow rate through each cage inlet port of a first design does not need to necessarily be equal to nonetheless be generally considered the same “first flow rate”. Moreover, in certain examples, flow rates described as, e.g., first and second flow rates can have overlapped or approximately equal flow rates and/or ranges of flow rates depending on various design parameters of the catheter 100 (such as, but not limited to, positioning and profile of the cage 200 and the flow profile of the catheter 100). Therefore, these configurations are fully within the spirit and scope of the presently disclosed technology.

By providing the first plurality of cage inlet ports 212 and the second plurality of cage inlet ports 222 upstream of the inlet ports 122 (relative to the flow into the cannula 112 through the inlet ports 122), a biomaterial (e.g., thrombus THR, it is noted that biomaterial hereafter is referred to as biomaterial THR, but can include biomaterial other than thrombus) is able to be captured by the cage 200 prior to entry into the cannula 112. Based on the size and positioning of the cage inlet ports 212, 222, the biomaterial THR can be configured to be captured in the first section 210, the second section 220, or both the first section 210 and the second section 220. In any scenario, the cage inlet ports 212, 222 are designed such that fluid is allowed to pass through, but not biomaterial THR greater than a predetermined size (which can inhibit operation of the pump assembly 108, as mentioned above).

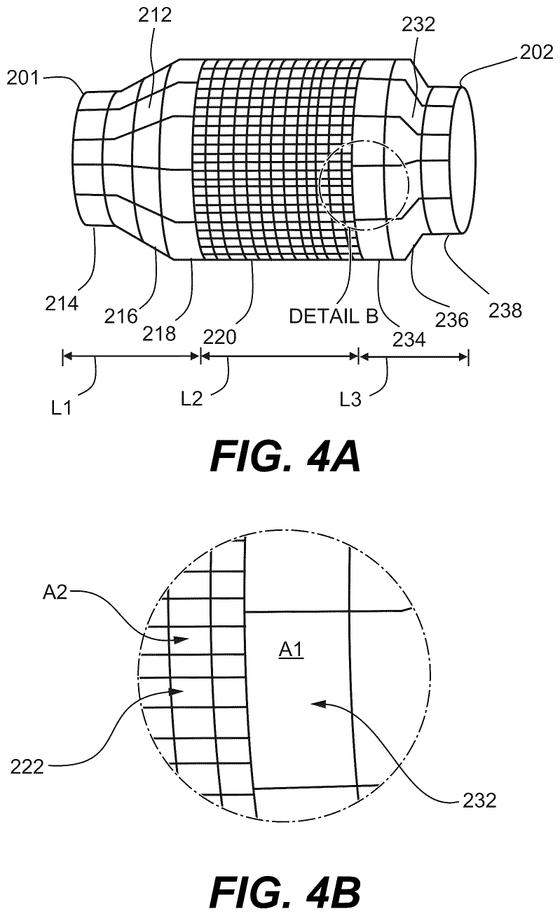

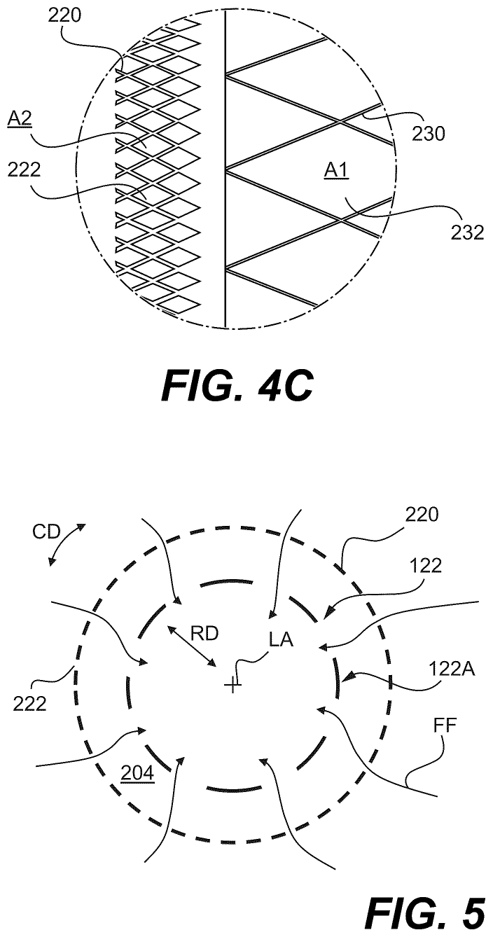

With specific reference to FIGS. 3 and 4A, the cage 200 can be formed partially or entirely from one or more mesh materials that comprise the above-mentioned cage inlet ports 212, 222, 232. The size of the respective inlet ports 212, 222, 232 may be selected based on, e.g., the desired flow profile. For example, the first cage inlet ports 212 may be larger than the second cage inlet ports 222 (discussed in greater detail with respect to FIGS. 4B and 4C). To achieve this, in some examples, one or more of the sections 210, 220, 230 may be formed separately from the others (e.g., the second section 220 could be a finer mesh material wrapped around either the inner or outer circumference of a mesh with larger openings, or the sections 210, 220, 230 could be separate mesh materials connected to one another).

As seen in FIGS. 4B and 4C, and as reference above, the mesh of the second section 220 may have a different design that of the first section 210 and the third section 230 (which can also be different or can be configured the same). In the presently illustrated example, the mesh of the first section 210 and the third section 230 both define respective cage inlet ports 212, 232 having a first area A1, whereas the mesh of the second section 220 defines cage inlet ports 222 having a second area A2 different from the first area A1. In this example, the second area A2 of each cage inlet port 222 is smaller than the first area A1 of each cage inlet port 212, 232 in the first and third sections 210, 230. The mesh inlet ports 212, 222, 232 can also take any appropriate shape, such as rectangular (FIG. 4B) or diamond (FIG. 4C) shapes. Besides modulating the flow rate(s), as discussed above, providing certain larger-sized inlet ports has other benefits, such as for guidewire access as well as blood washing.

Moreover, each section 210, 220, 230 extends a respective length L1, L2, L3 (FIG. 4A) along the longitudinal axis, which may be selected for similar reasons as that of the sizing of the cage inlet ports 212, 222, 232 (e.g., to achieve a desired flow profile). In some examples, along the first length L1, the first section 210 may include a linear section 214 that extends approximately parallel to the longitudinal axis LA and connects with the housing 113, an inclined section 216 that is sloped relative to the longitudinal axis LA, and another linear section 216 that extends approximately parallel to the longitudinal axis LA and connects with second section 220. Similarly, in some examples, along the third length L1, the first section 210 can include a linear section 238 that extends approximately parallel to the longitudinal axis LA and connects with the cannula 112, an inclined section 236 that is sloped relative to the longitudinal axis LA, and another linear section 234 that extends approximately parallel to the longitudinal axis LA and connects with second section 220.

As seen particularly in FIG. 5, due to the inclined sections 216, 236, an annular conduit 204 is defined between the second section 220 of the cage 200 and the inlet ports 122. The conduit 204 fluidically connects the inlet ports 122 and the cage inlet ports 212, 222, 232. The conduit spaces the second section 220 of the cage 200 from each inlet port 122 by at least a predetermined distance in the radial direction RD that is perpendicular to the longitudinal axis LA. In some examples, the predetermined distance can fall within the range of approximately a few microns (e.g., 5-10 microns) to approximately 3 millimeters. In other examples, the predetermined distance can be as large as approximately 1 centimeter. As those skilled in the art will appreciate, the predetermined distance can be adjusted to design for different flow rate requirements, as needed.

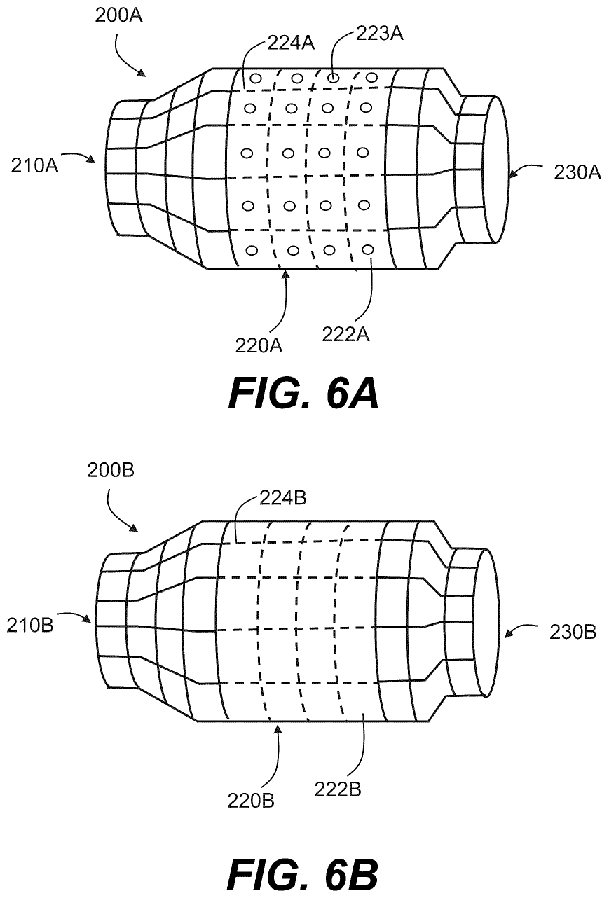

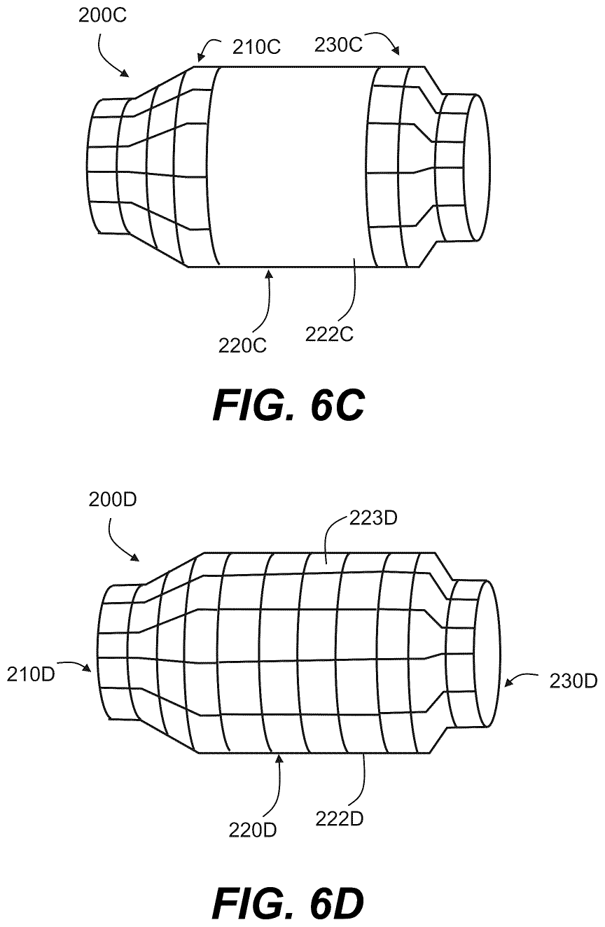

As mentioned above, the cage 200 (and, in general, the presently described system) are not necessarily limited to the configurations described in the foregoing paragraphs. Further to the above, FIGS. 6A-6D depict other exemplary configurations of the cage 200 in accordance with the presently disclosed technology.

FIG. 6A depicts an alternative configuration 200A of the cage 200. This alternative cage 200A has a similarly configured first section 210A and third section 230A compared with the previously described example, but with a different section 220A. The second section 220A of the alternative configuration 200A includes an intermediate mesh material 224A that connects the first section 210A and the second section 230 and a cuff 222A that, in use, extends around the inlet ports 122 in the circumferential direction CD. The cuff 222A connects to the intermediate mesh material 224A and can include cuff ports 223A defined therethrough in the radial direction RD that function in an equivalent manner as that of the cage inlet ports 222 of the first example. The cuff 222A can be made from any appropriate material, including a biocompatible metal or polymer material.

FIG. 6B depicts another alternative configuration 200B of the cage 200. This alternative cage 200B has a similarly configured first section 210B, second section 220B (with cuff 222B and intermediate mesh material 224B), and third section 230A compared with the previously described example 200A, but the cuff 222B does not include cuff ports. This design of the cuff 222B has a continuous annular shape and prevents fluid flow immediately adjacent the inlet ports 122.

FIG. 6C depicts yet another alternative configuration 200C of the cage 200. This alternative cage 200C has a similarly configured first section 210C, second section 220C (with cuff 222C), and third section 230C compared with the previously described examples 200A and 200B, but the cuff 222C does not include cuff ports and there is no intermediate mesh material that connects the first and third sections 210C, 230C. It will be appreciated that, of course, this configuration 200C of the cage 200 could include cuff ports similar to those shown in the example 200A of FIG. 6A.

FIG. 6D depicts yet another alternative configuration 200D of the cage 200. This alternative cage 200D has a similarly configured first section 210D, second section 220D (with intermediate mesh material 222D defining inlet ports 223D), and third section 230C compared with the previously described examples 200A. In this example 200D, the cuff is omitted, resulting in a cage 200D with approximately same-sized cage inlet ports in each section 210D, 220D, 230D.

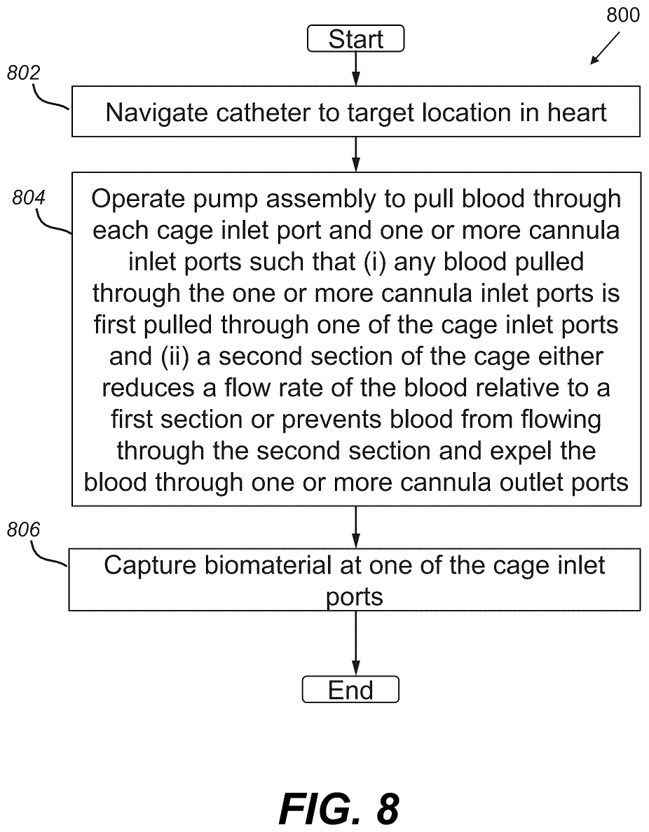

FIGS. 7A-7B illustrates an exemplary use case of the cage of the present disclosure. While the cage 200 of FIG. 3 is depicted, it will be appreciated by those skilled in the art that any of the foregoing cages 200A, 200B, 200C, 200D and/or other equivalent configurations can analogously employed without departing from the spirit and scope of the present disclosure. FIG. 8 illustrates an exemplary method 800 of performing a medical procedure in accordance with the present disclosure.

Making reference to FIGS. 7A-8 in conjunction with one another, the method 800 can include navigating 802 the catheter 100 to a target location (e.g., as shown in FIG. 1, where the target location is the pulmonary artery PA) in a heart HRT of a patient. In some examples, navigating 902 can include navigating through the superior vena cava SVC, through the right atrium RA, and through the pulmonary vein PV. As mentioned above, the catheter 100 can take any of the previously described forms, such as the catheter 100 depicted in FIG. 2. The pump assembly is operated 904 (see the rotational arrows in FIGS. 7A and 7B showing the rotor 109A spinning) to pull blood through each cage inlet port 212, 222, 232 (see the solid line arrows in FIGS. 7A and 7B showing blood being directed to and through the cage inlet ports 212, 222, 232) and the one or more inlet ports 122 such that (i) any blood pulled through the one or more inlet ports 122 is first pulled through one of the cage inlet ports 212, 222, 232 and (ii) the second section 220 of the cage 200 has a different flow profile relative to the first section 210 of the cage 200 (e.g., the second section 220 can have a reduced a flow rate of blood relative to the first section 210 or have blood prevented from flowing therethrough via, for example, a cuff). This is denoted by the first flow rate FFR and the second flow rate SFR labeling in FIGS. 7A-7B. As seen in FIG. 7B, the method 800 further can include capturing 906 the biomaterial THR at one of the cage inlet ports 212, 222, 232. While FIG. 7B depicts the biomaterial THR being captured at the first cage inlet ports 212 for the purposes of illustration, those skilled in the art will appreciate that the design of the cage 200 can be optimized to preferentially capture the biomaterial THR at any of the cage inlet ports 212, 222, 232, such as, but not limited to, the second cage inlet ports 222.

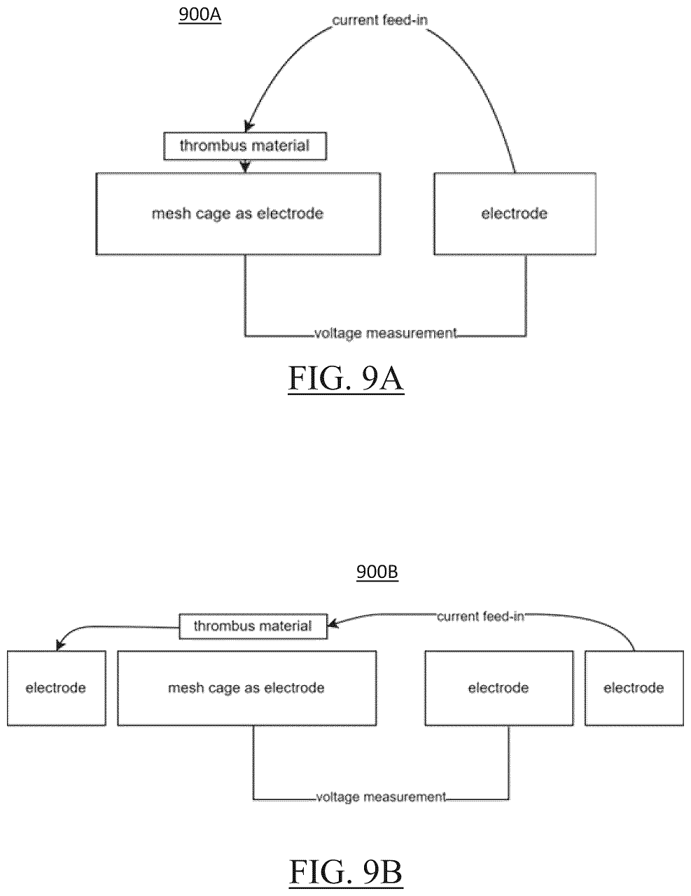

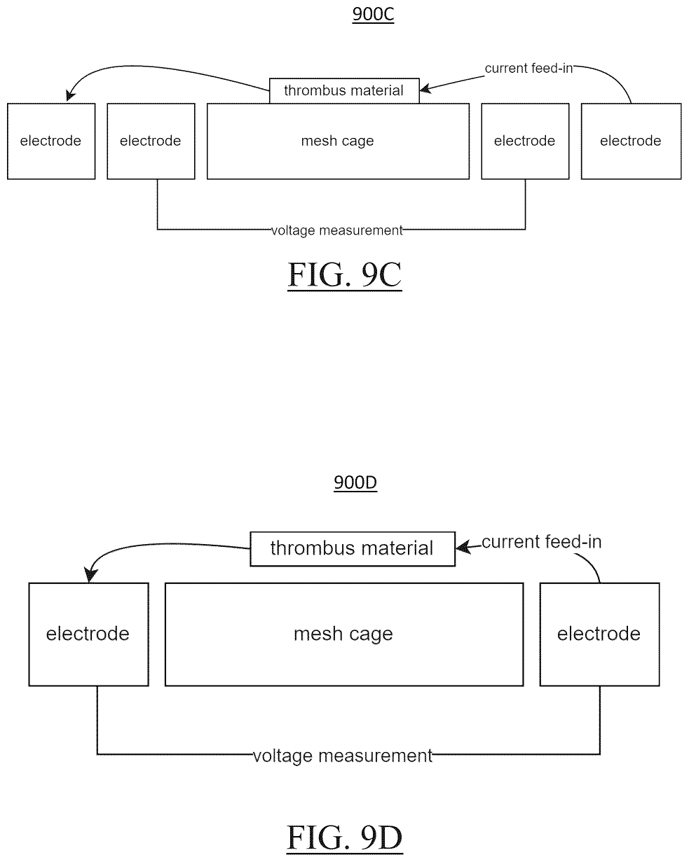

Making reference to FIGS. 9A-9D, and as mentioned above, various detection techniques for detecting capture of the biomaterial THR can be employed. For example, the catheter 100 can further include one or more sensors arranged near the cage inlet ports 212, 222, 232. This sensor can be used to detect the presence of the capture of biomaterial THR. As will appreciated by those skilled in the art, the sensor can be an optical sensor, a pressure sensor, or any other appropriate type of sensor. As will also be appreciated, the sensor may be positioned in any suitable location on the catheter 100.

In some examples, the cage 200 may be made of a conductive material and the sensor may be an electrode. See, for example, the exemplary schematic arrangement 900A depicted in FIG. 9A. The sensor may be located at a distance from the cage 200. In such examples, the cage 200 and the sensor may form an electrode pair. The electrode may generate an electric field. In such examples, the electrode pair may sense contact with tissues when the electric field is disrupted. The tissue may be biomaterial. For example, the electrode pair may be able to detect the presence of the capture of biomaterial THR. The electrode pair may be able to detect a change in measured impedance. As will be appreciated, any suitable number of electrode pairs may be used. See, for example, the exemplary schematic arrangement 900B depicted in FIG. 9B that illustrates two electrode pairs. The electrode pair may include a plurality of sensors. In such examples, the electrode pair may include two electrodes, the cage 200 and an electrode, or some combination of the two. The one or more electrodes may be ring-shaped. The one or more electrodes may be configured to cover at least a portion of the circumference of the cage 200 and/or one or more of the cage inlet ports. As discussed herein, the cage 200 may be made of a mesh material.

In other examples, the cage 200 may not be made of a conductive material. In such examples, the catheter 100 may include one or more electrode pairs. See, for example, the exemplary schematic arrangement 900C depicted in FIG. 9C that illustrates a four-electrode configuration and the exemplary schematic arrangement 900D depicted in FIG. 9D that illustrates a two-electrode configuration. As mentioned herein, the one or more electrodes may be ring-shaped. The one or more electrodes may be configured to cover at least a portion of the circumference of the cage 200 and/or one or more of the cage inlet ports. The one or more electrodes may all have the same geometry (e.g., all electrodes are ring-shaped). However, as will be appreciated, the one or more electrodes may have different geometries. The electrodes may be positioned at any suitable location along the length of the cage 200. In other examples, the electrodes may be positioned at any suitable location along the catheter 100.

The following clauses list non-limiting examples in accordance with the disclosed technology:
Clause 1. A catheter for insertion into a patient's vasculature, the catheter extending along a longitudinal axis and comprising: a catheter body comprising a distal end and a proximal end; a pump assembly disposed at the distal end of the catheter body and comprising a distal end portion; a cannula connected to the distal end portion of the pump assembly and comprising a proximal cannula portion and a distal cannula portion; one or more inlet ports disposed proximal the proximal cannula portion; one or more outlet ports disposed proximal the distal cannula portion; and a cage connected to the cannula and the pump assembly, extending along the longitudinal axis and in a circumferential direction about the longitudinal axis surrounding the one or more cannular inlet ports and comprising: a first section and a second section connected to the first section along the longitudinal axis, the first section being spaced from the one or more cannula input ports along the longitudinal axis and the second section being disposed over the one or more inlet ports in a radial direction; and a plurality of cage inlet ports comprising a first plurality of cage inlet ports disposed in the first section of the cage and a second plurality of cage inlet ports disposed in the second section of the cage, the pump assembly being operable to cause fluid to flow (i) through the first plurality of cage inlet ports at a first flow rate and through the second plurality of cage inlet ports at a second flow rate different from the first flow rate, (ii) through the one or more inlet ports, and (iii) through the one or more outlet ports.
Clause 2. The catheter of clause 1, the first plurality of cage inlet ports are configured to capture a biomaterial as the fluid flows therethrough.
Clause 3. The catheter of any one of clauses 1-2, wherein the first section comprises a first mesh comprising the first plurality of cage inlet ports.
Clause 4. The catheter of any one of clauses 1-3, wherein the second section comprises a second mesh comprising the second plurality of cage inlet ports.
Clause 5. The catheter of any one of clauses 1-4, wherein the second section does not comprise a mesh.
Clause 6. The catheter of any one of clauses 1-3 and 5, wherein the second section comprises a cuff extending around the one or more inlet ports in the circumferential direction.
Clause 7. The catheter of clause 6, wherein the cuff comprises a plurality of cuff ports that comprise the second plurality of cage inlet ports, the plurality of cuff ports being defined through the cuff in a radial direction.
Clause 8. The catheter of any one of clauses 1-7, wherein the second section extends along the longitudinal axis a second length.
Clause 9. The catheter of any one of clauses 1-8, wherein the cage further comprises a third section extending along the longitudinal axis from the second section and opposing the first section, the third section comprising a third mesh.
Clause 10. The catheter of clause 9, wherein the third mesh comprises a third plurality of cage inlet ports, each comprising a third area approximately equal to the first area.
Clause 11. The catheter of any one of clauses 9-10, wherein at least one of the first section or the third section comprises an inclined section that is sloped relative to the longitudinal axis to define an annular conduit between the cage and the one or more inlet ports.
Clause 12. The catheter of any one of clauses 1-11, wherein a proximal end of the cage is directly connected to the pump assembly and a distal end of the cage is directly connected to the proximal cannula portion of the cannula.
Clause 13. The catheter of clause 12, wherein the proximal end of the cage is directly connected to the pump assembly and the distal end of the cage is directly connected to the proximal cannula portion of the cannula via at least one of a mechanical connection or an adhesive connection.
Clause 14. The catheter of any one of clauses 1-13, wherein the one or more inlet ports are formed in a cannula inlet cage.
Clause 15. The catheter of any one of clauses 1-14, wherein the pump assembly is configured to pull fluid through the one or more inlet ports, through a lumen of the cannula, and expel the fluid out of the cannula through the one or more outlet ports.
Clause 16. The catheter of any one of clauses 1-15, wherein the pump assembly comprises: a rotor connected to the distal end of the catheter body; and one or more impeller blades connected to the rotor.
Clause 17. The catheter of clause 16, further comprising a motor connected to the rotor.
Clause 18. The catheter of any one of clauses 1-17, the cage comprising a biocompatible metal material.
Clause 19. A catheter for insertion into a patient's vasculature, the catheter extending along a longitudinal axis and comprising: a catheter body comprising a distal end and a proximal end; a pump assembly disposed at the distal end of the catheter body and comprising a distal end portion; a cannula connected to the distal end portion of the pump assembly and comprising a proximal cannula portion and a distal cannula portion; one or more inlet ports disposed proximal the proximal cannula portion; one or more outlet ports disposed proximal the distal cannula portion; and a cage connected to the cannula and the pump assembly, extending along the longitudinal axis and in a circumferential direction about the longitudinal axis surrounding the one or more cannular inlet ports and comprising: a first section and a second section connected to the first section along the longitudinal axis, the first section being spaced from the one or more cannula input ports along the longitudinal axis and the second section being disposed over the one or more inlet ports in a radial direction; and a plurality of cage inlet ports comprising: a first plurality of cage inlet ports disposed in the first section of the cage, each of the first plurality of cage inlet ports comprising a first area; and a second plurality of cage inlet ports disposed in the second section of the cage, each of the second plurality of cage inlet ports comprising a second area, the pump assembly being operable to cause fluid to flow (i) through the first plurality of cage inlet ports and through the second plurality of cage inlet ports, (ii) through the one or more inlet ports, and (iii) through the one or more outlet ports.
Clause 20. The catheter of clause 19, the first plurality of cage inlet ports are configured to capture a biomaterial as the fluid flows therethrough.
Clause 21. The catheter of any one of clauses 19-20, wherein the first section comprises a first mesh comprising the first plurality of cage inlet ports.
Clause 22. The catheter of any one of clauses 19-21, wherein the second section comprises a second mesh comprising the second plurality of cage inlet ports.
Clause 23. The catheter of any one of clauses 19-22, wherein the second section does not comprise a mesh.
Clause 24. The catheter of any one of clauses 19-21 and 23, wherein the second section comprises a cuff extending around the one or more inlet ports in the circumferential direction.
Clause 25. The catheter of clause 24, wherein the cuff comprises a plurality of cuff ports that comprise the second plurality of cage inlet ports, the plurality of cuff ports being defined through the cuff in a radial direction.
Clause 26. The catheter of any one of clauses 19-25, wherein the second section extends along the longitudinal axis a second length.
Clause 27. The catheter of any one of clauses 19-26, wherein the cage further comprises a third section extending along the longitudinal axis from the second section and opposing the first section, the third section comprising a third mesh.
Clause 28. The catheter of clause 27, wherein the third mesh comprises a third plurality of cage inlet ports, each comprising a third area approximately equal to the first area.
Clause 29. The catheter of any one of clauses 27-28, wherein at least one of the first section or the third section comprises an inclined section that is sloped relative to the longitudinal axis to define an annular conduit between the cage and the one or more inlet ports.
Clause 30. The catheter of any one of clauses 19-29, wherein a proximal end of the cage is directly connected to the pump assembly and a distal end of the cage is directly connected to the proximal cannula portion of the cannula.
Clause 31. The catheter of clause 30, wherein the proximal end is directly connected to the pump assembly and the distal end of the cage is directly connected to the proximal cannula portion of the cannula via at least one of a mechanical connection or adhesive connection.
Clause 32. A catheter for insertion into a patient's vasculature, the catheter extending along a longitudinal axis and comprising: a catheter body comprising a distal end and a proximal end; a pump assembly disposed at the distal end of the catheter body and comprising a distal end portion; a cannula connected to the distal end portion of the pump assembly and comprising a proximal cannula portion and a distal cannula portion; one or more inlet ports disposed proximal the proximal cannula portion; one or more outlet ports disposed proximal the distal cannula portion; and a cage connected to the cannula and the pump assembly, extending along the longitudinal axis and in a circumferential direction about the longitudinal axis surrounding the one or more cannular inlet ports and comprising: a first section and a second section connected to the first section along the longitudinal axis, the first section being spaced from the one or more cannula input ports along the longitudinal axis and the second section being disposed over the one or more inlet ports in a radial direction, the second section being configured to prevent fluid flow therethrough in the radial direction; and a plurality of cage inlet ports comprising a first plurality of cage inlet ports disposed in the first section of the cage, the pump assembly being operable to cause fluid to flow (i) through the first plurality of cage inlet ports, (ii) through the one or more inlet ports, and (iii) through the one or more outlet ports.
Clause 33. The catheter of clause 32, wherein the second section comprises a cuff extending around the one or more inlet ports in the circumferential direction.
Clause 34. The catheter of clause 33, wherein the cuff comprises a continuous annular shape with no ports defined in a radial direction.
Clause 35. The catheter of any one of clauses 33-34, wherein the cuff comprises an annular shape.
Clause 36. The catheter of any one of clauses 32-35, wherein the second section further comprises a second mesh at least one of wrapping around an outer circumference of the cuff or around an inner circumference of the cuff.
Clause 37. The catheter of any one of clauses 32-36, wherein the cuff comprises a biocompatible metal or biocompatible polymer material.
Clause 38. A medical system comprising: a catheter extending along a longitudinal axis and comprising: a catheter body comprising a distal end and a proximal end; a pump assembly disposed at the distal end of the catheter body and comprising a distal end portion; a cannula connected to the distal end portion of the pump assembly and comprising a proximal cannula portion and a distal cannula portion; one or more inlet ports disposed proximal the proximal cannula portion; one or more outlet ports disposed proximal the distal cannula portion; and a cage connected to the cannula and the pump assembly, disposed over the one or more inlet ports, and comprising a plurality of cage inlet ports, the cage extending along the longitudinal axis and in a circumferential direction about the longitudinal axis; and a controller connected to the proximal end of the catheter body and configured to operate the pump assembly to cause fluid to flow through the cage inlet ports, through the one or more inlet ports, and through the one or more outlet ports.
Clause 39. The medical system of clause 38, wherein the controller comprises a fluid reservoir that is configured to contain a purge fluid, the controller being configured to deliver the purge fluid through the pump assembly.
Clause 40. The medical system of clause 39, wherein the controller comprises a fluid supply control for controlling supply of the purge fluid to the pump assembly and a flow rate control for controlling operation of the pump assembly.
Clause 41. The medical system of any one of clauses 38-40, wherein the controller is configured to operate the pump assembly to pull a fluid through the one or more inlet ports, through a lumen of the cannula, and expel the fluid through the one or more outlet ports.
Clause 42. A method of performing a medical procedure comprising: navigating a catheter to a target location in a heart of a patient, the catheter extending along a longitudinal axis and comprising (i) a catheter body, (ii) a pump assembly connected to the catheter body, (iii) a cannula connected to the pump assembly and comprising one or more inlet ports, and (iv) a cage connected to the cannula and the pump assembly, the cage comprising a first section, a second section connected to the first section, and a plurality of cage inlet ports; operating the pump assembly to: pull blood through each cage inlet port and the one or more inlet ports such that (i) any blood pulled through the one or more inlet ports is first pulled through one of the cage inlet ports and (ii) the second section of the cage either reduces a flow rate of the blood relative to the first section or prevents blood from flowing through the second section; and capturing a biomaterial at one of the cage inlet ports.
Clause 43. The method of clause 42, wherein the target location comprises the pulmonary artery of the heart.
Clause 44. The method of any one of clauses 42-43, wherein navigating the catheter to the target location comprises navigating through the superior vena cava, through the right atrium, and through the pulmonary vein.
Clause 45. The method of any one of clauses 42-44, wherein the biomaterial comprises a thrombus.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the disclosed technology, which are within the spirit of the disclosure or equivalent to the subject matter found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

What is claimed is:

1. A catheter for insertion into a patient's vasculature, the catheter extending along a longitudinal axis and comprising:
a catheter body comprising a distal end and a proximal end;
a pump assembly disposed at the distal end of the catheter body and comprising a distal end portion;
a cannula connected to the distal end portion of the pump assembly and comprising a proximal cannula portion and a distal cannula portion;
one or more inlet ports disposed proximal the proximal cannula portion;
one or more outlet ports disposed proximal the distal cannula portion; and
a cage connected to the cannula and the pump assembly, extending along the longitudinal axis and in a circumferential direction about the longitudinal axis surrounding the one or more cannular inlet ports and comprising:
a first section and a second section connected to the first section along the longitudinal axis, the first section being spaced from the one or more cannula input ports along the longitudinal axis and the second section being disposed over the one or more inlet ports in a radial direction; and
a plurality of cage inlet ports comprising a first plurality of cage inlet ports disposed in the first section of the cage and a second plurality of cage inlet ports disposed in the second section of the cage,
the pump assembly being operable to cause fluid to flow (i) through the first plurality of cage inlet ports at a first flow rate and through the second plurality of cage inlet ports at a second flow rate different from the first flow rate, (ii) through the one or more inlet ports, and (iii) through the one or more outlet ports.

2. The catheter of claim 1, the first plurality of cage inlet ports are configured to capture a biomaterial as the fluid flows therethrough.

3. The catheter of claim 1, wherein the first section comprises a first mesh comprising the first plurality of cage inlet ports.

4. The catheter of claim 1, wherein the second section comprises a second mesh comprising the second plurality of cage inlet ports.

5. The catheter of claim 1, wherein the second section does not comprise a mesh.

6. The catheter of claim 1, wherein the second section comprises a cuff extending around the one or more inlet ports in the circumferential direction.

7. The catheter of claim 1, wherein the second section extends along the longitudinal axis a second length.

8. The catheter of claim 1, wherein the cage further comprises a third section extending along the longitudinal axis from the second section and opposing the first section, the third section comprising a third mesh.

9. The catheter of claim 1, wherein a proximal end of the cage is directly connected to the pump assembly and a distal end of the cage is directly connected to the proximal cannula portion of the cannula.

10. The catheter of claim 1, wherein the one or more inlet ports are formed in a cannula inlet cage.

11. The catheter of claim 1, wherein the pump assembly is configured to pull fluid through the one or more inlet ports, through a lumen of the cannula, and expel the fluid out of the cannula through the one or more outlet ports.

12. The catheter of claim 1, wherein the pump assembly comprises:
a rotor connected to the distal end of the catheter body; and
one or more impeller blades connected to the rotor.

13. The catheter of claim 1, the cage comprising a biocompatible metal material.

14. A catheter for insertion into a patient's vasculature, the catheter extending along a longitudinal axis and comprising:
a catheter body comprising a distal end and a proximal end;
a pump assembly disposed at the distal end of the catheter body and comprising a distal end portion;
a cannula connected to the distal end portion of the pump assembly and comprising a proximal cannula portion and a distal cannula portion;
one or more inlet ports disposed proximal the proximal cannula portion;
one or more outlet ports disposed proximal the distal cannula portion; and
a cage connected to the cannula and the pump assembly, extending along the longitudinal axis and in a circumferential direction about the longitudinal axis surrounding the one or more cannular inlet ports and comprising:
a first section and a second section connected to the first section along the longitudinal axis, the first section being spaced from the one or more cannula input ports along the longitudinal axis and the second section being disposed over the one or more inlet ports in a radial direction; and
a plurality of cage inlet ports comprising:
a first plurality of cage inlet ports disposed in the first section of the cage, each of the first plurality of cage inlet ports comprising a first area; and
a second plurality of cage inlet ports disposed in the second section of the cage, each of the second plurality of cage inlet ports comprising a second area,
the pump assembly being operable to cause fluid to flow (i) through the first plurality of cage inlet ports and through the second plurality of cage inlet ports, (ii) through the one or more inlet ports, and (iii) through the one or more outlet ports.

15. The catheter of claim 14, the first plurality of cage inlet ports are configured to capture a biomaterial as the fluid flows therethrough.

16. The catheter of claim 14, wherein the first section comprises a first mesh comprising the first plurality of cage inlet ports.

17. The catheter of claim 14, wherein the second section comprises a second mesh comprising the second plurality of cage inlet ports.

18. The catheter of claim 14, wherein the second section does not comprise a mesh.

19. The catheter of claim 14, wherein the second section comprises a cuff extending around the one or more inlet ports in the circumferential direction.

20. A medical system comprising:
a catheter extending along a longitudinal axis and comprising:
a catheter body comprising a distal end and a proximal end;
a pump assembly disposed at the distal end of the catheter body and comprising a distal end portion;
a cannula connected to the distal end portion of the pump assembly and comprising a proximal cannula portion and a distal cannula portion;
one or more inlet ports disposed proximal the proximal cannula portion;
one or more outlet ports disposed proximal the distal cannula portion; and
a cage connected to the cannula and the pump assembly, disposed over the one or more inlet ports, and comprising a plurality of cage inlet ports, the cage extending along the longitudinal axis and in a circumferential direction about the longitudinal axis; and
a controller connected to the proximal end of the catheter body and configured to operate the pump assembly to cause fluid to flow through the cage inlet ports, through the one or more inlet ports, and through the one or more outlet ports.
